(19) United States
(12) Patent Application Publication
(10) Pub. No.: US 2025/0025064 A1
(43) Pub. Date: Jan. 23, 2025

(54) BREATHING EFFORT SENSING APPARATUSES AND METHODS

(71) Applicant: REVEAL BIOSENSORS, INC., San Jose, CA (US)

(72) Inventor: Guy Meredith HATCH, Logan, UT (US)

(73) Assignee: REVEAL BIOSENSORS, INC., San Jose, CA (US)

(21) Appl. No.: 18/713,866

(22) PCT Filed: Nov. 30, 2022
(86) PCT No.: PCT/US22/80612

§ 371 Date: May 28, 2024

Related U.S. Application Data
(60) Provisional application No. 63/265,034, filed on Dec. 7, 2021.

(51) Int. Cl. A61B 5/08; A61B 5/00; A61B 5/0205; A61B 5/024; A61B 90/00

(52) U.S. Cl. CPC A61B 5/0816; A61B 5/02405; A61B 5/02438; A61B 5/02444; A61B 5/0803; A61B 5/441; A61B 5/4836; A61B 5/6824; A61B 5/6831; A61B 90/06; A61B 5/0205; A61B 2090/064; A61B 2560/0214; A61B 2560/0443; A61B 2562/0233

(57) ABSTRACT

Devices and methods are described for continuous and non-invasive sensing of: (1) breathing effort, and (2) cellular oxygen need vs. cellular oxygen supply. The devices configurable for remote patient care configured to monitor one or more of sleep disordered breathing, hypertension, heart failure, pneumonia, asthma, chronic obstructive pulmonary disease (COPD), respiratory distress syndrome (RDS) in premature infants, and acute respiratory distress syndrome (ARDS) in children and adults. Additionally, the devices are configurable to control delivery of therapy.

### CROSS-REFERENCE

This application claims the benefit of U.S. Provisional Application No. 63/265,034, filed Dec. 7, 2021, entitled BREATHING EFFORT SENSING APPARATUS AND METHODS which application is incorporated herein in its entirety by reference.

### BACKGROUND

Many illnesses and injuries involve compromise of lung function. Therapy for these conditions typically includes both airway pressure breath cycle assistance to breathing to assure sufficient ventilation and removal of CO₂ and additional oxygen in the breathing gas to normalize the intake of oxygen via compromised lungs. Modern breathing assistance devices, beginning with the ‘iron lung’ for victims of paralytic polio, saved lives by providing fixed breathing rates and fixed volumes of breathing gas exchange per minute. However, with this approach, the patient's internal regulation of the breathing process could only be approximated, with the assumption that physiologic adaptation would occur if the match was close enough. Manual adjustments of breathing assistance settings are currently made by clinicians are primarily based on periodic blood gas measurements and on continuous pulse oximetry and end-tidal CO₂ monitoring. The resulting therapy tends to be more supportive than needed in order to avoid a crisis if the patient's condition worsens, but increases risk of therapy-associated lung injury, or barotrauma, and increases difficulty during weaning from breathing assistance. Current monitoring and therapy devices have saved many lives that would likely have been lost. However, despite providing a wealth of information about their operation, modern breathing assistance systems still require clinician skill and time to manually adjust settings. Limitations of the currently available biometric information reduce the accuracy and timeliness of regulation of ventilation and oxygen supply, potentially resulting in tissue injuries from excess pressure, damage to vital organs from excessive or too-rapidly increased oxygen supply, and difficulty weaning from breathing assistance.

More complete and more timely biometric information could enable automated regulation of breathing assistance, which could, in turn, shorten the duration of treatment, improve outcomes by reducing risk of therapy-related injuries, and by reducing the risk and cost of hospital readmission. Past attempts to improve therapy by automating control of breathing assistance have been hampered by not having a non-invasive, objective indicator of breathing effort, and by lack of awareness of cellular oxygen supply vs., oxygen need. Recent discovery of new biometric information indicating the level of effort with each breath and the patient's cellular oxygen status offers the possibility of improving breathing assistance therapy by automating its regulation.

Currently, modern medical breathing assistance techniques target a minimum duration of therapy leading to normal, unassisted breathing as the adverse process resolves. To fully accomplish this goal, the level of breathing assistance needs to be accurately adjusted relative to the amount of effort the patient is making, and provide exactly the right amount of oxygen supplementation by: maintaining adequate ventilation; avoiding deconditioning of breathing muscles; avoiding compromise of breathing regulation; maintaining adequate oxygen intake while avoiding hyperoxic injury; and correlating breathing data with pulse amplitude, heart rate, and heart rate variability.

Minute ventilation is the volume of breathing gas exchanged through the lungs per minute. One of the key objectives of assisted breathing is to supplement the mechanical effort needed to achieve the needed minute ventilation rate; but not to entirely take over unless absolutely necessary. To enable this desired balance, the breathing effort exerted by the patient to overcome the elastic recoil of the lungs and resistance to airflow into and from the lungs must be continuously and objectively monitored; preferably non-invasively. Existing monitoring of thoracic electrical impedance, or of the change of circumference of the chest and abdomen, indicate the motion of breathing; not the effort. Esophageal manometry can measure breathing effort, but is invasive, uncomfortable, and stressful to the patient. Providing less than the needed level of mechanical support to patients with compromised breathing risks respiratory and cardiac arrest, but providing more than needed support risks lung injury and deconditioning of breathing muscles; both of which may compromise or prevent weaning from support.

Breathing muscles need to continue to work at, or slightly above, their normal resting work load to maintain strength and conditioning. Additionally, breathing assistance that bypasses or distorts the patient's natural chemical sensor and central nervous system regulation of breathing rate and depth risks compromise or loss of function of these vital control processes.

Insufficient biometric information and the need for manual ventilator control combine to make breathing assistance therapy one of the most challenging aspects of modern intensive care. Moreover, regulation of breathing rate and depth can also be compromised by pain control medications and may not be adequately mature in premature newborn infants. Therefore, the adequacy of the patient's breathing control system needs to be accurately assessed during initial and interim evaluations. Adjusting support to normalize blood gas and pulse oximetry values often results in distortion or suppression of natural breathing regulation, making decreasing and discontinuing support more difficult. The adequacy of cellular oxygen supply cannot currently be measured or monitored. The currently accepted alternatives include measuring blood oxygen saturation by laboratory ‘blood gas’ analysis of invasively sampled arterial blood, and non-invasive monitoring by pulse oximetry. Monitoring blood oxygen, however, has not enabled avoidance of ‘oxidative stress’ tissue injuries. Additionally, each person has a unique, ‘normal’ level of resting feedback control of their vital functions. The clinical challenge is to identify the patient's ‘normal’ resting level, and then keep the patient at the ‘slightly stressed’ upper margin of their ‘normal’ to prevent deconditioning of internal control mechanisms. The information needed to accomplish these goals includes continuous biometric information about breathing effort and cellular oxygen need vs., cellular oxygen supply.

### SUMMARY

Disclosed are devices and methods for continuous and non-invasive sensing of: (1) breathing effort, and (2) cellular oxygen need vs., cellular oxygen supply. Current and future patient breathing assistance needs may be better met by automated regulation of the fraction of inspired oxygen (FiO₂) and breathing cycle timing, pressure and flow based on continuous, non-invasive monitoring of breathing effort and cellular oxygen need vs., cellular oxygen supply. Additionally disclosed are devices configurable for remote patient care configured to monitor one or more of sleep disordered breathing, hypertension, heart failure, atrial fibrillation, and chronic lung diseases including asthma and chronic obstructive pulmonary disease (COPD). The disclosed devices and methods monitor cellular hypoxic stress, breathing rate and effort, pulse amplitude variation, heart rate, body orientation and activity, and skin temperature. The disclosed devices are configurable to communicate with a software application (“app”) on a mobile device, such as a cell phone, and transmit information to a central location such as a hospital, doctor's office, or home patient monitoring program.

Devices are configurable to include motion-tolerant sensors and skin pigment tolerant optical sensors. The devices are conformable to the user/patient in contact with a skin surface, and have a sufficiently long battery life.

Both the foregoing general description and the following detailed description are exemplary and explanatory only and are not restrictive of the disclosed embodiments, as claimed.

### INCORPORATION BY REFERENCE

All publications, patents, and patent applications mentioned in this specification are herein incorporated by reference to the same extent as if each individual publication, patent, or patent application was specifically and individually indicated to be incorporated by reference.
US 2017/0049336 A1 to Hatch for Physiological Sensors, Systems, Kits and Methods Therefor published Feb. 23, 2017;
US 2020/008531 A1 to Hatch for Energy Conversion Monitoring Devices, Systems and Methods;
U.S. Pat. No. 4,838,257 A to Hatch issued Jun. 13, 1989 for Ventilator;
U.S. Pat. No. 5,830,137 A to Scharf issued Nov. 3, 1998 for Green Light Pulse Oximeter;
U.S. Pat. No. 6,801,799 B2 to Mendelson, issued Oct. 5, 2004, for Pulse Oximeter and Method of Operation;
U.S. Pat. No. 7,691,067 B2 to Westbrook, issued Apr. 6, 2010, for Method for Measuring Central Venous Pressure or Respiratory Effort;
U.S. Pat. No. 7,738,935 B1 to Turcott, issued Jun. 15, 2010, for Methods and Devices for Reduction of Motion-Induced Noise in Pulse Oximetry;
U.S. Pat. No. 8,073,516 B2 to Scharf issued Dec. 6, 2011, for Separating Motion from Cardiac Signals Using Second Order Derivative of the Photo-Plethysmogram and Fast Fourier Transforms;
U.S. Pat. No. 8,133,176 B2 to Porges, issued Mar. 13, 2012, for Method and Circuit for Indicating Quality and Accuracy of Physiological Measurements;
U.S. Pat. No. 8,346,327 B2 to Campbell, issued Jan. 1, 2013, for Method for Identification of Sensor Site by Local Skin Spectrum Data;
U.S. Pat. No. 9,125,563 B2 for “Signal Monitoring System Including EMI-Shielding Coupler,” CAS Medical Systems, Inc., Sep. 8, 2015;
U.S. Pat. No. 9,357,954 B2 for “Simultaneous Measurement of Pulse and Regional Blood Oxygen Saturation,” Covidien LP, issued Jun. 7, 2016;
U.S. Pat. No. 10,327,710 B2 for “Age Calibration for Tissue Oximetry,” Nonin Medical, Inc., issued Jun. 25, 2019;
U.S. Pat. No. 10,335,072 B2 for “Physiologic Monitor,” Masimo Corporation, issued Jul. 2, 2019;
U.S. Pat. No. 10,369,310 B2 for “Method and Apparatus for Treatment of Respiratory Disorders” ResMed Limited issued Aug. 6, 2019;
U.S. Pat. No. 10,376,670 B2 for “Methods and Systems for Sleep Management” ResMed Sensor Technologies issued Aug. 13, 2019;
U.S. Pat. No. 10,383,569 B2 for “Mechanical Ventilation in the Presence of Sleep Disordered Breathing” ResMed Limited, issued Aug. 20, 2019;
U.S. Pat. No. 10,638,960 to Hatch for Optical Physiologic Sensors and Methods published Apr. 27, 2017; and
WO 2015/168235 A1 to Hatch published Nov. 5, 2015, for Physiological Sensors, Systems, Kits and Methods Therefor.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A is a diagram showing a polygraph trace with breaths and cuff pulses captured over time with a 5 second interval marked;

FIG. 1B is an image of cross-section of an upper arm of a user with a device according to the disclosure held in surface contact with the upper arm by an arm band;

FIGS. 1C-D are 3D model images of an arm band-mounted device according to the disclosure;

FIG. 1E illustrates a block diagram of a breathing effort sensor apparatus in communication with a network, or directly to an electronic device;

FIG. 2 is a data recording obtained during 8 hours of normal sleep breathing;

FIG. 3 is a 26-minute segment of adult sleep recording of Cellular Energy Index illustrating severe sleep disordered breathing with mixed Obstructive Sleep Apnea (OSA) and Central Sleep Apnea (CSA);

FIGS. 4A-D conceptually illustrate the recorded intensity of intermittently detected infrared light during breath periods, FIG. 4A conceptually depicts the moderate increase of detected infrared intensity value that occurs during a normal breath, FIG. 4B conceptually depicts an extreme increase in detected infrared intensity value during increased breathing effort, FIG. 4C conceptually depicts the minimal-to-no variation in detected infrared intensity value during central apnea, and FIG. 4D conceptually depicts decrease in detected infrared intensity during a ventilator driven breath that more than overcomes the elastic recoil of the lungs and/or resistance to gas flow of the patient's airway;

FIG. 5 provides a conceptual graph illustrating the correlation of a plurality of measurable biometrics of physiologic stress through the minimum-to-maximum range of stress;

FIG. 6 is a flow diagram for relating monitored breathing effort and other biometrics relative to the level of breathing assistance;

FIG. 7 is a flow diagram for automatically regulating the fraction of inspired oxygen (FiO₂) based on monitoring of Cellular Energy Index 700 rather than by manual adjustment based on blood gas measurements and/or pulse oximetry; and

FIG. 8 conceptually depicts the arm band tension sensing system, including exposed and cross-section views of the internal mechanism.

### DETAILED DESCRIPTION

Light absorption by the skin of the upper arm has been found to vary in synchrony with the normal breathing cycle, and to robustly indicate the relative effort of each breath. The decreased intrathoracic pressure during inhalation that produces flow of air into the lungs also enhances the return of venous blood from the skin of the upper arm toward the heart, reducing absorption of the sensor's infrared light and resulting in increased intensity of the detected light after the light has diffused through the skin. Normal, unlabored breathing produces a moderate rise in detected infrared light intensity with each breath. No breathing effort, such as during central apnea, produces little to no change of detected infrared intensity. Profoundly increased inspiratory effort, such as during severe airway or lung disease, produces larger than normal amplitude increases in detected infrared intensity with each breath effort. This real-time sensor data can be used to continuously regulate the rate of inflation gas flow and the level of pressure provided by the medical breathing assistance system, such that the effort of breathing is maintained just slightly greater than normal at rest to assure breathing muscles are kept in tone. U.S. Pat. No. 4,838,257 A describes a ventilator pressure and flow regulation system capable of providing this method of breathing assistance.

Breathing effort data can also be used to indicate the patient's internal breathing drive governing breathing rate and depth. Dysfunction of the patient's blood CO₂ sensor in the base of the brain, and to a lesser extent, blood oxygen sensors in the carotid arteries may also adversely affect weaning from breathing assistance therapy. Less difficulty during weaning from support is the main anticipated benefit of continuously synchronizing breathing assistance to the patient's natural drive.

A cellular energy monitor provides insight into cellular adaptation to variations in oxygen supply, potentially resulting in advances in the safety and effectiveness of breathing assistance and several modern medical therapies. Automating regulation of breathing oxygen supply based on Cellular Energy monitoring also offers potentially safer and more effective therapy than can be achieved with manual adjustment based on pulse oximetry monitoring and/or periodic blood gas measurements.

The addition of pulse amplitude monitoring on the upper arm provides monitoring of heart rate, heart rate variability, pulse amplitude variation, and pulsus paradoxus that can be factored together with monitoring of breathing effort and cellular oxygen status to provide a combined index trend of these primary vital functions. The desired ‘slightly stressed’ physiologic status during assisted breathing is defined herein as: (1) slightly elevated heart rate, with (2) decreased heart rate variability, and (3) increased pulse peak amplitude, increased pulsus paradoxus, and increased mean pulse amplitude compared with these vital parameters when the patient is fully relaxed.

An automated therapy process to recognize and achieve the desired level of slightly increased stress could start with the level of ventilation assistance being raised until the patient becomes fully relaxed and lets the system do all the work; e.g., the breath effort trend decreases to flatline and pulsus paradoxus diminishes. The system would then slowly reduce the breathing assistance peak inspiratory pressure and/or assisted breath rate per minute until the breath effort and pulsus paradoxus trends rise to their respective slightly higher than normal levels, accompanied by slightly elevated heart rate, decreased heart rate variability, and increased pulse peak and mean pulse amplitude compared with the fully relaxed status. Operation at this level of breathing assistance would continue for a period of time, then the evaluation process could be partially repeated to reassess the patient's disease status. In the event that monitored breathing effort and pulsus paradoxus increases, indicating increased severity of lung disease or increased breathing distress, the level of breathing assistance would be increased to reestablish the desired level of monitored breathing effort and pulsus paradoxus. Ultimately, this automated cycle of evaluation and adjusted breathing assistance will lead to the discontinuation of external support when the patient is able to sustain adequate ventilation and breathing control on their own with only slight breathing and physiologic distress.

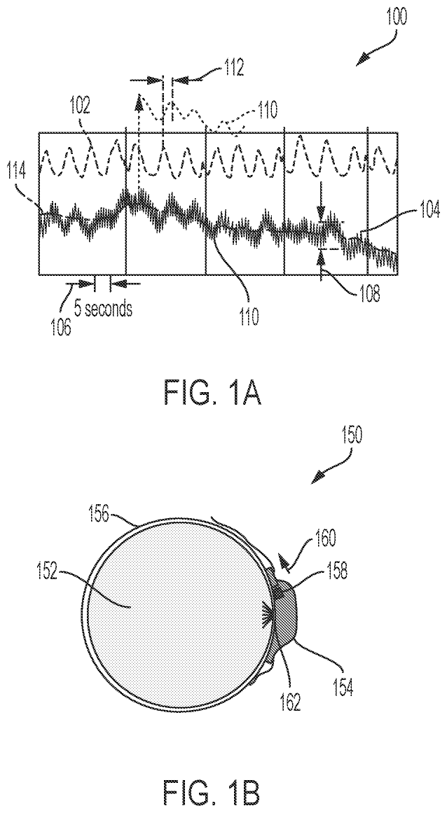

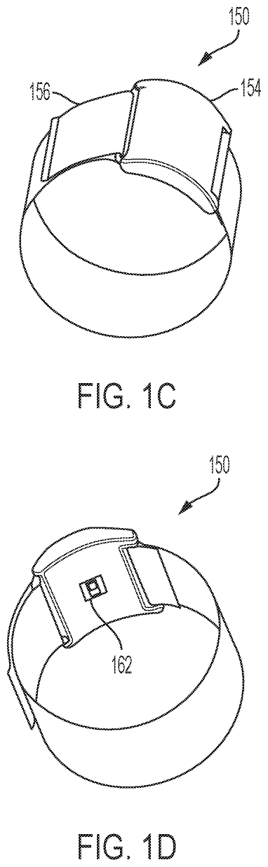

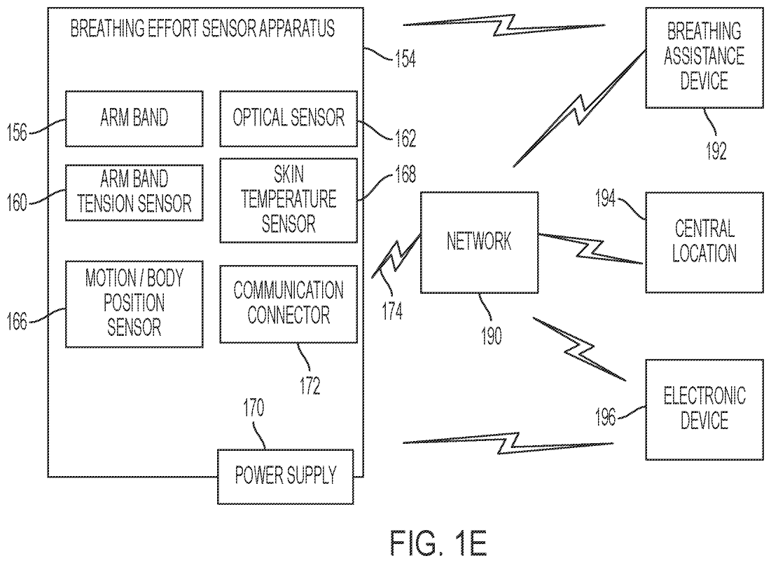

FIG. 1A is a diagram showing a segment of a forensic polygraph trace 100 with breaths 102 and pneumatic arm cuff pulses 104 recorded over time with a 5 second interval marked 106. Features in this data include pulse amplitude 108, mean pulse amplitude, or pulsus paradoxus 110, breath 102 to mean pulse amplitude 110 variation phase delay 112, and average pulse amplitude 114. The upper trace is provided for clearer illustration.

FIG. 1B is a cross-section view of the upper arm 152 of a user with a breathing effort sensor apparatus having a housing 154 in communication with an arm band 156, where the housing 154 is held in surface contact with the upper arm by the arm band 156. The housing 154 has an upper surface, a lower surface opposite the upper surface, and four side walls that define an interior cavity for housing the device components. The upper surface and lower surface can be curved. For example, the exterior facing surface of the lower surface can be concave to achieve optimal contact with the arm surface. The upper surface can also be curved. In one configuration the curved upper surface can have a similar curve such that it is concave from the interior facing surface and convex from the interior facing surface at a value identical to the curve of the lower surface. Alternatively, the upper surface can have a larger curve so that, for example, it creates a dome. Extending from the opposing side of the sensor housing is an arm band tension-sensing clip that engages the other end of the armband 156. The armband clip, in turn, engages a force sensor, such as a piezo stack or a load cell sensor, to electro-mechanically detect the tension between the two ends of the armband. Once the breathing effort sensor apparatus and arm band are secured to the user's arm, a baseline arm band tension is determined. As the tension changes from the baseline, the changes in tension are detected by the tension sensor.

A pair of apertures 162 is provided on the skin-facing side of the housing. One end of the arm band engages a mating feature of the sensor housing. The pulse amplitude 108 and variation of mean pulse amplitude 110 shown in FIG. 1A are measured by a biometric sensor 158 in the breathing effort sensor apparatus 154 as heart rate-cyclic variations in tension 160 of the armband, in a manner analogous to how the pneumatic cuff pressure pulse sensor is used in forensic polygraph recording 100. Pulse detection by this method would also enable continuous monitoring of heart rate and heart rate variability. The sample forensic polygraph trace in FIG. 1A shows a normal variation in mean pulse amplitude 110 in synchrony with breathing, also known as pulsus paradoxus. The trace also shows a rising and falling average pulse amplitude trend 114 over time that is known to be reliably analogous to variations in mean arterial blood pressure, such as could be invasively measured with an intra-arterial catheter and electro-mechanical transducer. The forensic polygraph method uses these variations in average pneumatic cuff pulse amplitude as a continuous, non-invasive analog of mean arterial pressure to indicate the varying tone of the sympathetic nervous system that is, in turn, driven by the level of emotional stress induced in the test subject by either telling the truth or lying. The disclosed monitoring application similarly uses the variations in average pulse amplitude to indicate changes in physiologic stress, such as induced by sleep disordered breathing (SDB), which results in abnormal lack of episodes of ‘dipping’ blood pressure during sleep. Lack of ‘dipping’ of blood pressure during sleep correlates with increased risk of developing high blood pressure. Screening for ‘non-dippers’ is not currently included in sleep lab SDB diagnostic studies. Reduction of hypertension risk and possibly resolution of established hypertension would be potential benefits of detecting ‘non-dipping’ during sleep in a sleep lab or at home, followed by sensor-guided optimization of SDB therapy to re-establish ‘dipping.’ Various disease processes and injuries, and corresponding medical therapies, also produce variations in mean blood pressure. Providing clinicians with greater awareness of these variations could potentially also be beneficial in the diagnosis and therapy for a wide variety of other conditions.

An optical cellular energy monitor sensor 162 is also held in direct contact with the skin of the upper arm to detect the varying absorption of red and infrared light. Analysis of this spectral absorption data is used to indicate cellular oxygen supply status, breathing rate, and breathing effort. The arm band 156 is looped around a tension sensor clip 166 and adjusted and attached back upon itself, such as with hook-loop (e.g., Velcro©) 168.

FIGS. 1C-D are 3D model images of an arm band-mounted breathing effort sensor apparatus according to the disclosure. The arm band-mounted breathing effort sensing apparatus comprises: a housing with an upper surface, a lower surface and four side walls defining an interior chamber. The inner chamber is configured to house the electronics for the apparatus. An armband is provided that is securable to the housing. The armband can be removably securable to allow for different sized armbands to be used for the apparatus to accommodate different sized patients. An optical sensor partially positioned within the housing of the breathing effort sensing apparatus is also provided. The optical sensor has a sensing surface facing external to the breathing effort sensing apparatus. A tension sensor is positioned within the housing and receives tension information from the armband when the apparatus is in use. A processor can also be provided that is positioned within the housing and configured to receive information from the optical sensor and the tension sensor. The processor can also be configured to determine one or more of cellular oxygen supply breathing rate and breathing effort. Additionally, a transmitter can be provided that is positioned within the housing configured to receive information from the processor and transmit the information and/or instructions to an external device. The breathing effort sensing apparatus is configurable to monitor a plurality of events including, but not limited to: heart rate, heart rate variability, and pulse amplitude. The breathing effort sensing apparatus ca also derive one or more of, for example, a mean pulse amplitude and a pulsus paradoxus variation. The power source for the apparatus can be removable and/or rechargeable.

FIG. 1E is a block diagram of the breathing effort sensor apparatus 154. Components of the breathing effort sensor apparatus 154 can include an arm band 156, an arm band tension sensor 160, an optical sensor 162, a motion and body position sensor 166, a skin temperature sensor 168, and a power supply 170. The power supply 170 can be a battery having a long life, such a replaceable battery, or a permanent, rechargeable battery. An internal power supply, such as an internal battery, can also be used. The internal power supply can be incorporated so that it is not removable and/or accessible outside the apparatus. The internal power supply is particularly suitable the apparatus is fully watertight apparatus, thus preventing the internal components from being exposed to fluid including, sweat, water, etc. Power can also be provided or recharged via a USB connector on a USB port.

A wired communication connector 172 may optionally be provided, such as for wired communication with a secondary device, such as a breathing assistance device 192. In most configurations, the breathing effort sensor apparatus 154 is in wireless communication 174, e.g., via a network 190 to other devices or locations, or directly, such as to a breathing assistance device 192. The data collected from the breathing effort sensor apparatus 154, can be sent to a breathing assistance device 192, such as a CPAP or mechanical ventilator, to provide biometric feedback, which can then be used to guide automatic or semi-automatic adjustment of the therapeutic delivery by the breathing assistance device. The data collected from the breathing effort sensor apparatus 154 can also be sent to a central location 194, such as a doctor's office, hospital, and the like. The data collected from the breathing effort sensor apparatus 154 can also be sent to an electronic device 196, such as a cell phone or tablet.

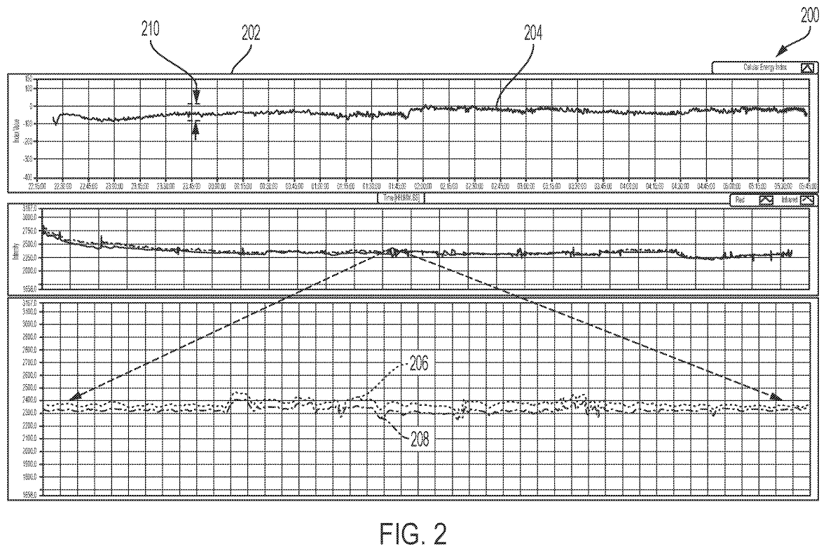

FIG. 2 is a data recording 200 obtained during 8 hours of normal sleep breathing. The upper graph 202 depicts the derived Cellular Energy index trend 204 obtained by subtracting the detected intensity value of the infrared light 206 from the detected intensity value of the red light 208 when each are sampled once per second. The red and infrared intensity data trends are depicted in the middle graph on the same time scale as the top graph, and time-expanded in the bottom data graph. It has been observed, empirically, that the Cellular Energy Index trend during non-stressed adult sleep varies between about +20 down to about-75 in value 210.

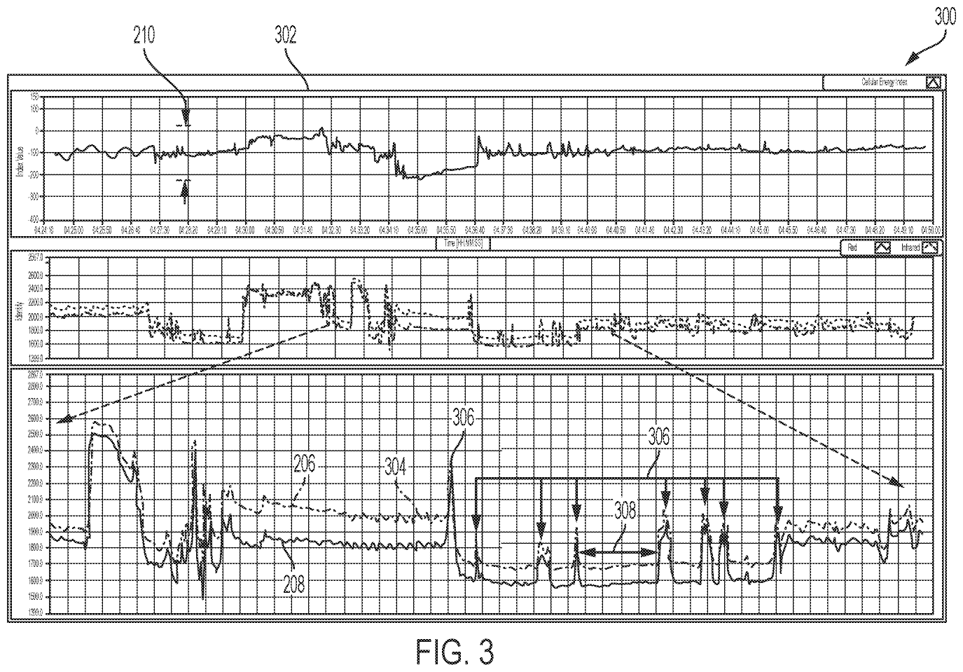

FIG. 3 is a 26-minute portion of an adult sleep data recording 300 of Cellular Energy Index 302 illustrating severe sleep disordered breathing with mixed obstructive sleep apnea (OSA) and central sleep apnea (CSA). Regular, normal breaths 304 are best detected in the infrared raw data trace. There are also recorded episodes of extreme breathing effort during airway obstruction 306 at the beginning of, and recurring during a three-minute period of alternating central apnea 308 and airway obstruction events 306. The detected intensity value trend of the infrared light 206 is shown, along with the detected intensity value trend of the red light 208. In contrast with the normal sleep breathing data depicted in FIG. 2, the Cellular Energy Index trend in this recording segment deviated 210 much more deeply into the negative, or skin cellular hypoxia, range.

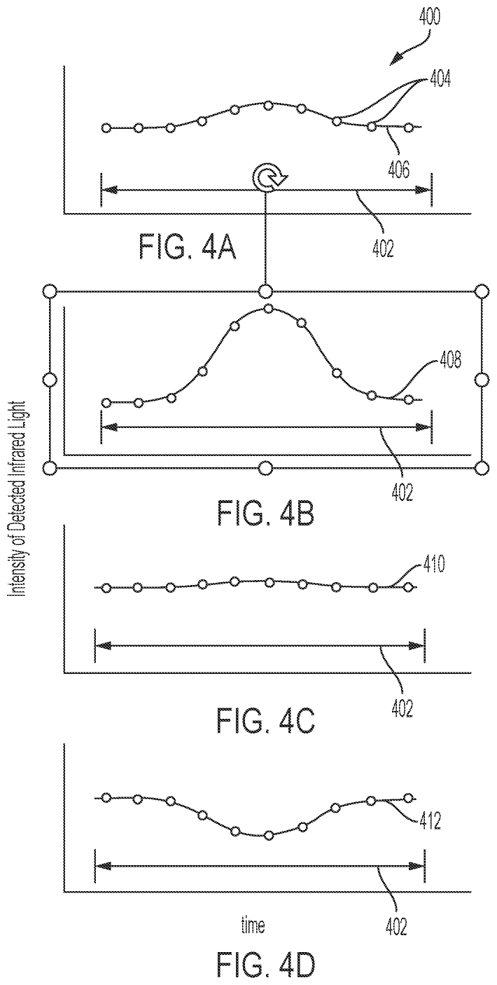

FIGS. 4A-D conceptually illustrate the recorded intensity of detected infrared light during breath periods 402, with detected intensity on the y-axis and time on the x-axis. Experience indicates that infrared absorption by the skin, as indicated by detected intensity sampling 404, may need to occur at up to 10 Hz to minimize aliasing of the breathing waveform data during rapid breathing; such as with infants. As conceptually shown in FIG. 4A there is a moderate increase of detected infrared intensity value during a normal breath 406 during the breath period 402. FIG. 4B conceptually depicts a more pronounced positive deviation in detected infrared intensity value with increased breathing effort 408 during the breath period 402, such as during an obstructed airway event during sleep, or during labored breathing due to asthma, pneumonia, or other lung impairment. FIG. 4C conceptually depicts the minimal to no variation in detected infrared intensity value 410 during central apnea during the breath period 402. FIG. 4D conceptually depicts decreased detected infrared intensity, such as would occur during a ventilator-assisted breath 412 that more than overcomes the elastic recoil of the lungs and/or air flow resistance of the patient's airway during the breath period 402. This is a recognizable indication of excessive breathing assistance that may be associated with increased risk of lung tissue injury and of suppression or distortion of the patient's natural breathing drive.

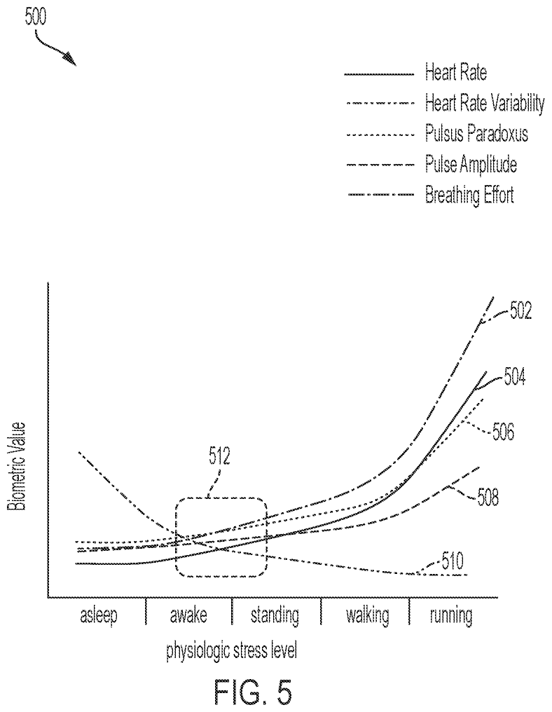

FIG. 5 provides a conceptual graph 500 illustrating a correlation of a plurality of measurable biometrics of physiologic stress, with the biometric value on the vertical axis and increasing physiologic stress on the horizontal axis. The addition of continuous monitoring of breathing effort 502 via the disclosed breathing effort sensor apparatus provides a robust new indicator for guiding regulation of ventilation. Heart rate 504, pulsus paradoxus 506, average pulse amplitude 508, and heart rate variability 510 respond in consistent patterns relative to the level of physiologic stress, and can now be continuously and non-invasively monitored and displayed as trends. As a result of enhanced monitoring achieved with the disclosed breathing effort sensor apparatus, the listed biometrics can be monitored to provide a recognizable zone of ‘slightly increased stress’ 512 that can then be used as the therapy control target for automatically regulating ventilation support via the ventilation support devices at all levels of disease severity.

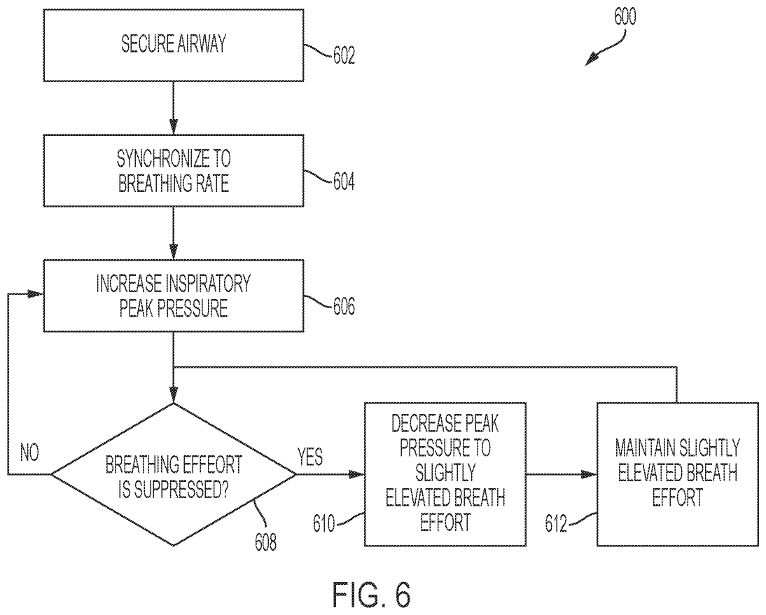

FIG. 6 is a flow diagram for relating monitored breathing effort 600 and other biometrics relative to the therapeutic level of breathing assistance. The age and size of the patient, the diagnosis, and the severity of the breathing distress will determine the need and timing of establishing a secure airway 602, such as an endotracheal tube. The combination of monitoring ventilator breathing gas flow and airway pressure, and monitoring the biometrics of the patient, will help to characterize the initial, naturally-controlled breathing rate of the patient to which the mechanical assistance will be synchronized 604. Beginning at a modest peak inspiratory pressure setting, a programmed, step-wise process of gradually increasing peak inspiratory pressure will then begin, with each step evaluated 606 relative to the resulting decrease of the patient's monitored index of breathing effort. This process continues until monitored breathing effort is suppressed 608, then the peak inspiratory pressure is gradually stepped down until the desired level of breathing effort and the desired levels of the biometric indicators of physiologic stress are reached 610. Changes in monitored breathing effort can then provide automatic guidance to continuously regulate peak inspiratory pressure to remain within the desired zone 610. As the patient's breathing distress resolves, the system automatically weans ventilation support to the point where clinical judgment can determine when the secured airway can be safely removed.

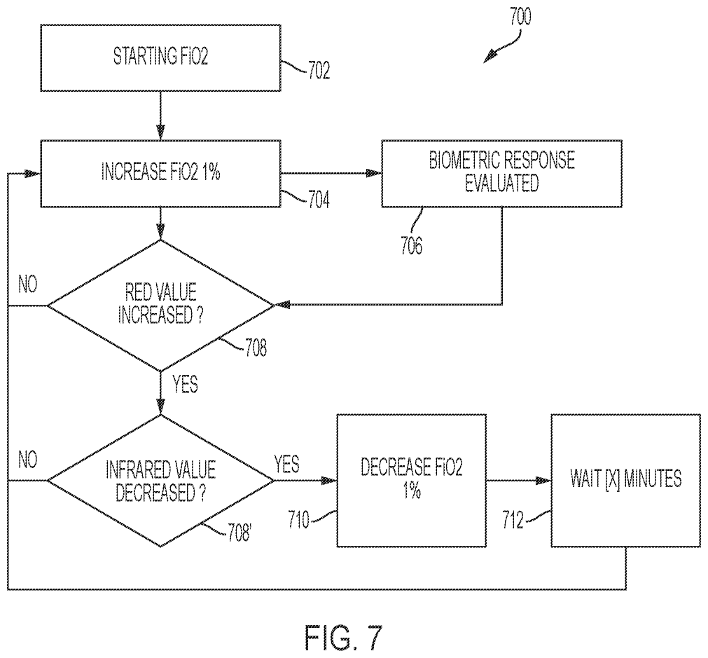

FIG. 7 is a flow diagram for regulating the fraction of inspired oxygen (FiO₂) based on monitoring of cellular energy index 700, rather than, or in addition to, blood gas measurements and/or pulse oximetry. It is assumed that the clinicians caring for premature newborn infants will likely administer intra-tracheal surfactant immediately following birth to help normalize the infant's lung mechanics. The anticipated status of cellular adaptation to oxygen supply will determine the starting FiO₂ level 702. In the case of premature newborn infants, this starting FiO₂ will be based on the combination of the gestational age at birth and the amount of fetal distress observed prior to delivery. Based on our new insights regarding cellular adaptation to low oxygen supply, the highest risk (e.g., less than 30 weeks gestation, with fetal distress prior to birth) premature newborn infants will likely need to start at an FiO₂ of 0.05 (i.e., 5% Oxygen, 95% Nitrogen). Full term newborn infants with minimal evidence of fetal distress could start at FiO₂ of 0.10 (i.e., 10% Oxygen, 90% Nitrogen) to initially match the normal ppO₂ of oxygen supplied via their placenta. Once Cellular Energy index monitoring is established, the FiO₂ is increased by 0.01 (1%) 704 and the resulting biometric response is evaluated 706. Compressed oxygen is currently blended with compressed air in a pressure-balanced gas mixing valve assembly that is typically manually controlled. Alternatively, pulse-width modulation (PWM) electronic control of a compressed oxygen source valve could be used to add varying-duration pulses of oxygen to a flow of compressed air or nitrogen to vary the fraction of oxygen in the delivered breathing gas. Using nitrogen and oxygen could provide a less than atmospheric oxygen level breathing gas blend, as mentioned above. The Cellular Energy Index, the detected red intensity, and the detected infrared intensity trends are then used as biometric feedback for automated control of the delivered FiO₂. If the detected intensity of the red signal increases, and the detected intensity of the infrared signal does not decrease 708′, the FiO₂ is increased 0.01 (1%) 704. However, if the infrared signal intensity decreases 708′ with an increase in FiO₂ (i.e., the cellular hyperoxia threshold of the infant's skin has been reached) the FiO₂ will be decreased by 0.01 (1%) to avoid cellular hyperoxia and resulting risk of ‘oxidative stress’ injuries 710. The wait time ‘X’ between FiO₂ level updates 712 will target the time constant of the biometric response 706, which may be unique with each patient and likely will vary with the patient's age and size. Preliminary research evidence and known physiologic principles identify vasoconstriction in the skin, and resulting measurable (with Cellular Energy monitoring) cellular hypoxia in the skin, as the human body's first response to hypoxic stress; consistently prior to detection of a decrease in blood oxygen saturation with pulse oximetry monitoring or arterial blood gas measurement.

It logically follows that skin cells would be the body's most-adapted tissue in response to episodes of decreased oxygen supply, making the skin the most relevant and responsive tissue for monitoring oxygen supply status. It is also anticipated that the oxygen need of newborn infants will increase over time, up to or possibly above the oxygen supplied by breathing atmospheric air with FiO₂=0.208 if they have impaired lung function. If there is no increase in red signal and no decrease in infrared signal, the FiO₂ will be increased by 0.01 (1%) 704. This regulation process will maintain the infant's oxygen intake at or above the oxygen supply that was received prior to birth via their placenta, and below the oxygen supply level where cellular hyperoxia is likely to trigger adhesion of leukocytes to the microvasculature endothelium of immature vital organs. Current use of atmospheric or higher FiO₂ continues to be associated with potentially devastating injuries to premature infant eyes, brain, gut, and failure of the ductus arteriosus to naturally close. As with ischemia/reperfusion injury (IRI), such as during reperfusion therapy for ischemic stroke, heart attack, and implantation of transplant organs, a rapid increase in oxygen supply is known to be associated with leukocyte-endothelial surface adhesion-induced microvascular obstruction of blood flow (ischemia), and tissue damage.

Similar microvascular obstruction-induced pathology may also be associated with a too-abrupt increase in oxygen supply in the breathing gas during resuscitation of children and adult victims of respiratory or cardiac arrest. In these cases, vital organ blood vessels have likely adapted to lower oxygen supply, making the starting FiO₂ of 0.1 (10% Oxygen, 95% Nitrogen) likely beneficial for them as well.

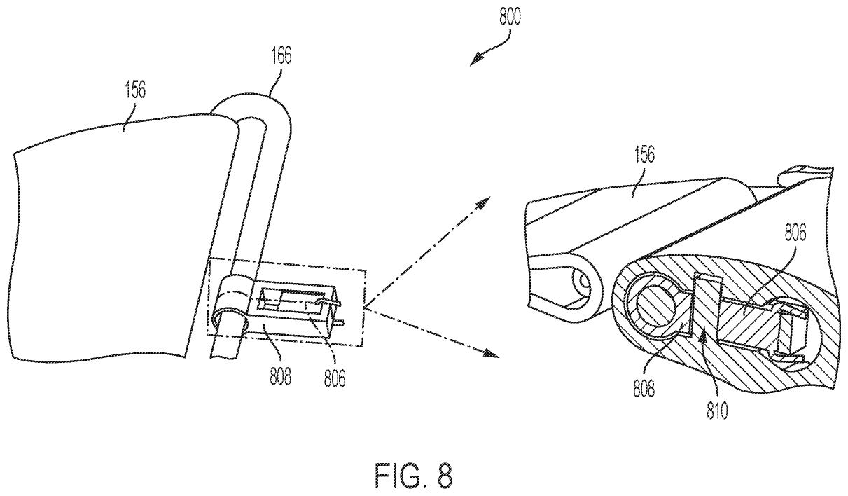

FIG. 8 conceptually depicts the arm band tension sensing system. The arm band 156 is looped around the captured tension sensor clip 166. In the preferred embodiment using a piezo-stack sensor 806, the tension sensor clip passes through a tension sensor mount frame 808 that mechanically converts arm band tension to compression of the piezo-stack sensor 806. The tension sensor 806 and sensor mount frame 808 are retained within the monitor housing 154 by a protrusion of the housing 810. Tension of the armband 156 produces a compression force on the piezo-stack sensor, which generates a piezoelectric voltage output that is used to electronically monitor the subtle, cyclic variations in the armband tension generated by the pulsation of the brachial artery in the upper arm.

The systems and methods according to aspects of the disclosed subject matter may utilize a variety of computer and computing systems, communications devices, networks and/or digital/logic devices for operation. Each may, in turn, be configurable to utilize a suitable computing device which can be manufactured with, loaded with and/or fetch from some storage device, and then execute, instructions that cause the computing device to perform a method according to aspects of the disclosed subject matter.

A computing device can include without limitation a mobile user device such as a mobile phone, a smart phone and a cellular phone, a personal digital assistant (“PDA”), such as an iPhone®, a tablet, a laptop and the like. In at least some configurations, a user can execute a browser application over a network, such as the Internet, to view and interact with digital content, such as screen displays. A display includes, for example, an interface that allows a visual presentation of data from a computing device. Access could be over or partially over other forms of computing and/or communications networks. A user may access a web-browser, e.g., to provide access to applications and data and other content located on a web-site or a web-page of a web-site.

A suitable computing device may include a processor to perform logic and other computing operations, e.g., a stand-alone computer processing unit (“CPU”), or hard wired logic as in a microcontroller, or a combination of both, and may execute instructions according to its operating system and the instructions to perform the steps of the method, or elements of the process. The user's computing device may be part of a network of computing devices and the methods of the disclosed subject matter may be performed by different computing devices associated with the network, perhaps in different physical locations, cooperating or otherwise interacting to perform a disclosed method. For example, a user's portable computing device may run an app alone or in conjunction with a remote computing device, such as a server on the Internet. For purposes of the present application, the term “computing device” includes any and all of the above discussed logic circuitry, communications devices and digital processing capabilities or combinations of these.

Certain embodiments of the disclosed subject matter may be described for illustrative purposes as steps of a method which may be executed on a computing device executing software. Included are software program code/instructions that can be provided to the computing device or at least abbreviated statements of the functionalities and operations performed by the computing device in executing the instructions. Some possible alternate implementation may involve the function, functionalities and operations occurring out of the order, including occurring simultaneously or nearly so, or in another order or not occurring at all. Aspects of the disclosed subject matter may be implemented in parallel or seriatim in hardware, firmware, software or any combination(s) of these, co-located or remotely located, at least in part, from each other, e.g., in arrays or networks of computing devices, over interconnected networks, including the internet, and the like.

The instructions may be stored on a suitable “machine readable medium” within a computing device or in communication with or otherwise accessible to the computing device. As used in the present application a machine readable medium is a tangible storage device and the instructions are stored in a non-transitory way. At the same time, during operation, the instructions may at sometimes be transitory, e.g., in transit from a remote storage device to a computing device over a communication link. However, when the machine readable medium is tangible and non-transitory, the instructions will be stored, for at least some period of time, in a memory storage device, such as a random access memory (RAM), read only memory (ROM), a magnetic or optical disc storage device, or the like, arrays and/or combinations of which may form a local cache memory, e.g., residing on a processor integrated circuit, a local main memory, e.g., housed within an enclosure for a processor of a computing device, a local electronic or disc hard drive, a remote storage location connected to a local server or a remote server access over a network, or the like. When so stored, the software will constitute a “machine readable medium,” that is both tangible and stores the instructions in a non-transitory form. At a minimum, therefore, the machine readable medium storing instructions for execution on an associated computing device will be “tangible” and “non-transitory” at the time of execution of instructions by a processor of a computing device and when the instructions are being stored for subsequent access by a computing device.

As will be appreciated by those skilled in the art, the disclosed devices can use wireless networks that incorporate a variety of types of mobile devices, such as, e.g., cellular and wireless telephones, PCs (personal computers), laptop computers, wearable computers, cordless phones, pagers, headsets, printers, PDAs, etc. For example, mobile devices may include digital systems to secure fast wireless transmissions of voice and/or data. Typical mobile devices include some or all of the following components: a transceiver (for example a transmitter and a receiver, including a single chip transceiver with an integrated transmitter, receiver and, if desired, other functions); an antenna; a processor; display; one or more audio transducers (for example, a speaker or a microphone as in devices for audio communications); electromagnetic data storage (such as ROM, RAM, digital data storage, etc., such as in devices where data processing is provided); memory; flash memory; and/or a full chip set or integrated circuit; interfaces (such as universal serial bus (USB), coder-decoder (CODEC), universal asynchronous receiver-transmitter (UART), phase-change memory (PCM), etc.). Other components can be provided without departing from the scope of the invention.

Additionally, wireless LANs (WLANs) in which a mobile user can connect to a local area network (LAN) through a wireless connection may be employed for wireless communications. Wireless communications can include communications that propagate via electromagnetic waves, such as light, infrared, radio, and microwave. There are a variety of WLAN standards that currently exist, such as Bluetooth®, IEEE 802.11, and the obsolete HomeRF. Bluetooth products may be used to provide links between mobile computers, mobile phones, portable handheld devices, personal digital assistants (PDAs), and other mobile devices and connectivity to the Internet. Bluetooth is a computing and telecommunications industry specification that details how mobile devices can easily interconnect with each other and with non-mobile devices using a short-range wireless connection. Bluetooth creates a digital wireless protocol to address end-user problems arising from the proliferation of various mobile devices that need to keep data synchronized and consistent from one device to another, thereby allowing equipment from different vendors to work seamlessly together.

Wireless network devices may include, but are not limited to Bluetooth devices, WiMAX (Worldwide Interoperability for Microwave Access), Multiple Interface Devices (MIDs), 802.11x devices (IEEE 802.11 devices including, 802.11a, 802.11b and 802.11g devices), HomeRF (Home Radio Frequency) devices, Wi-Fi devices, GPRS (General Packet Radio Service) devices, 3 G cellular devices, 2.5 G cellular devices, GSM (Global System for Mobile Communications) devices, EDGE (Enhanced Data for GSM Evolution) devices, TDMA type (Time Division Multiple Access) devices, or CDMA type (Code Division Multiple Access) devices, including CDMA2000. Each network device may contain addresses of varying types including but not limited to an IP address, a Bluetooth Device Address, a Bluetooth Common Name, a Bluetooth IP address, a Bluetooth IP Common Name, an 802.11 IP Address, an 802.11 IP common Name, or an IEEE MAC address.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the claims define the scope of the invention and that methods and structures within the scope of these claims and their equivalents be covered thereby.

What is claimed:

1. A breathing effort sensing apparatus comprising:
a housing with an upper surface, a lower surface and four side walls defining an interior chamber;
an armband securable to the housing;
an optical sensor partially positioned within the housing of the breathing effort sensing apparatus with a sensing surface of the optical sensor facing external to the breathing effort sensing apparatus;
a tension sensor positioned within the housing wherein the tension sensor receives tension information from the armband;
a processor positioned within the housing configured to receive information from the optical sensor and the tension sensor;
a transmitter positioned within the housing configured to receive information from the processor and transmit the information to an external device; and
a power source.

2. The breathing effort sensing apparatus of claim 1 wherein the optical sensor is configurable to detect absorption of red light and infrared light.

3. The breathing effort sensing apparatus of claim 1 wherein the processor is configured to determine one or more of a cellular oxygen supply, a breathing rate, and a breathing effort.

4. The breathing effort sensing apparatus of claim 1 configured to monitor pulse timing to derive one or more of a heart rate and a heart rate variability.

5. The breathing effort sensing apparatus of claim 1 configured to monitor pulse amplitude to derive one or more of a mean pulse amplitude and a pulsus paradoxus variation.

6. The breathing effort sensing apparatus of claim 1 wherein the power source is one or more of removable and rechargeable.

7. The breathing effort sensing apparatus of claim 1 wherein the transmitter is configured to transmit data to a breathing assistance device.

8. The breathing effort sensing apparatus of claim 1 wherein the transmitter is configured to transmit an instruction to a breathing assistance device.

9. The breathing effort sensing apparatus of claim 1 wherein the transmitter is configured to transmit data to one or more of a central location and an electronic device.

10. A networked breathing effort sensing apparatus comprising:
a housing with an upper surface, a lower surface and four side walls defining an interior chamber;
an armband securable to the housing;
an optical sensor partially positioned within the housing of the breathing effort sensing apparatus with a sensing surface of the optical sensor facing external to the breathing effort sensing apparatus;
a tension sensor positioned within the housing wherein the tension sensor receives tension information from the armband;
a processor positioned within the housing configured to receive information from the optical sensor and the tension sensor;
a transmitter positioned within the housing configured to receive information from the processor and transmit the information to an external device; and
a power source,
wherein the networked breathing effort sensing apparatus is in communication with one or more of breathing assistance device, a central location, and an electronic device.

11. The networked breathing effort sensing apparatus of claim 10 wherein the optical sensor is configurable to detect absorption of red light and infrared light.

12. The networked breathing effort sensing apparatus of claim 10 wherein the processor is configured to determine one or more of a cellular oxygen supply, a breathing rate, and a breathing effort.

13. The networked breathing effort sensing apparatus of claim 10 configured to monitor pulse timing to derive one or more of a heart rate and a heart rate viability.

14. The networked breathing effort sensing apparatus of claim 10 wherein the power source is one or more of removable and rechargeable.

15. The networked breathing effort sensing apparatus of claim 10 wherein data is transmitted to the breathing assistance device.

16. The networked breathing effort sensing apparatus of claim 10 wherein instructions are transmitted to the breathing assistance device.

17. The networked breathing effort sensing apparatus of claim 10 wherein data is transmitted to one or more of the central location and the electronic device.

18. A method of detecting a fraction of inspired oxygen (FiO₂) comprising:
providing a breathing effort sensing apparatus comprising a housing with an upper surface, a lower surface and four side walls defining an interior chamber, an armband securable to the housing, an optical sensor partially positioned within the housing of the breathing effort sensing apparatus with a sensing surface of the optical sensor facing external to the breathing effort sensing apparatus, a tension sensor positioned within the housing wherein the tension sensor receives tension information from the armband, a processor positioned within the housing configured to receive information from the optical sensor and the tension sensor, a transmitter positioned within the housing configured to receive information from the processor and transmit the information to an external device, and a power source;
determining a starting FiO₂ level;
increasing an FiO₂ level by a target amount;
evaluating a biometric response to the increased FiO₂ level, wherein the step of evaluating includes determining
if the red light signal increases and the infrared light signal does not decrease, increase the FiO₂ level and monitor the biometric response to the increased FiO₂,
if the infrared light signal intensity decreases with an increase in FiO₂ then decrease the FiO₂ level a target amount and monitor the biometric response to the decreased FiO₂;
if there is no increase in the red light signal and no decrease in the infrared light signal the FiO₂ will be increased by the target amount, and
if the infrared light value does not decrease, increase the FiO₂ by the target amount, and wait for a period of time and monitor the biometric response to the increased FiO₂; and
repeating the step of evaluating the biometric response until the breathing effort sensing apparatus is turned off.

19. The method of detecting FiO₂ according to claim 18 wherein the target amount of increase of FiO₂ is 1%.

20. The method of detecting FiO₂ according to claim 18 wherein the target amount of decrease of FiO₂ is 1%.

21. A method of operating a breathing assistance device in combination with a breathing effort sensing apparatus comprising:
providing a breathing effort sensing apparatus comprising a housing with an upper surface, a lower surface and four side walls defining an interior chamber, an armband securable to the housing, an optical sensor partially positioned within the housing of the breathing effort sensing apparatus with a sensing surface of the optical sensor facing external to the breathing effort sensing apparatus, a tension sensor positioned within the housing wherein the tension sensor receives tension information from the armband, a processor positioned within the housing configured to receive information from the optical sensor and the tension sensor, a transmitter positioned within the housing configured to receive information from the processor and transmit the information to an external device, and a power source;
providing a breathing assistance device in communication with the breathing effort sensing apparatus;
detecting an absorption of red light and an absorption of infrared light via the breathing effort sensing apparatus;
determining one or more of a cellular oxygen supply, a breathing rate, and a breathing effort via the breathing effort sensing apparatus; and
transmitting data from the breathing effort sensing apparatus to the breathing assistance device.

22. The method of operating a breathing assistance device in combination with a breathing effort sensing apparatus of claim 21 wherein the data transmitted from the breathing effort sensing apparatus includes instructions to control the breathing assistance device.

23. The method of operating a breathing assistance device in combination with a breathing effort sensing apparatus of claim 21 wherein the data transmitted from the breathing effort sensing apparatus is processed by the breathing assistance device to control operation of the breathing assistance device.

24. The method of operating a breathing assistance device in combination with a breathing effort sensing apparatus of claim 21, further comprising the step of transmitting the data to one or more of a central location and an electronic device.
